(19) United States
(12) Patent Application Publication
(10) Pub. No.: US 2026/0275238 A1
(43) Pub. Date: Sep. 17, 2026

(54) COMPOSITIONS AND METHODS AND USES RELATING THERETO

(71) Applicant: INNOSPEC LIMITED, Ellesmere Port (GB)

(72) Inventors: Delia GALINDO, Winston-Salem, NC (US); Stephen Moss O'CONNOR, Davidson, NC (US); Maurizio PERONI, Castiglione delle Stiviere (IT); Alessandra ANDREOLI

(21) Appl. No.: 19/157,000

(22) PCT Filed: Mar. 3, 2023
(86) PCT No.: PCT/EP2024/055505

§ 371 Date: Aug. 15, 2025

Related U.S. Application Data
(60) Provisional application No. 63/449,668, filed on Mar. 3, 2023.

(30) Foreign Application Priority Data
Mar. 20, 2023 (GB) 23162990.8

(51) Int. Cl. C11D 1/94; C11D 1/12; C11D 1/28; C11D 1/66; C11D 1/83; C11D 1/90; C11D 17/04

(52) U.S. Cl. CPC C11D 1/94; C11D 1/83; C11D 17/042; C11D 1/126; C11D 1/28; C11D 1/662; C11D 1/90

(57) ABSTRACT

The present invention relates to detergent compositions. In particular the invention relates to liquid or solid detergent compositions for use in toilet care, manual dishwashing, automatic dishwashing, laundry, fabric care, kitchen care, carpet cleaning, air fresheners, vehicle care, polishing products, machine cleaning and maintenance, pesticides, insecticides, fungicides, herbicides, oilfield chemical applications, marine applications, personal care and institutional/industrial cleaning. The detergent compositions may be especially useful in household cleaning applications, for example for use in laundry.

The present invention relates to detergent compositions. In particular the invention relates to liquid or solid detergent compositions for use in toilet care, manual dishwashing, automatic dishwashing, laundry, fabric care, kitchen care, carpet cleaning, air fresheners, vehicle care, polishing products, machine cleaning and maintenance, pesticides, insecticides, fungicides, herbicides, oilfield chemical applications, marine applications, personal care and institutional/industrial cleaning. The detergent compositions may be especially useful in household cleaning applications, for example for use in laundry.

Detergent compositions are commonly known for household use, particularly for laundry and dishwashing applications. Such compositions may be provided in loose powder or liquid form where a user measures out a dose each time before use.

Many consumers prefer to buy detergent compositions in unit dose form in which the dose is pre-measured and a single tablet or pouch is added to the washing machine or dishwasher. Unit dose detergent compositions are often provided in the form of compressed powder tablets or pouches comprising liquids encapsulated in a water-soluble polymer.

Environmental concerns are becoming increasingly important in relation to consumer products, with many consumers seeking to choose products which have a reduced environmental impact compared to well-established products. It is therefore highly desirable to provide detergent compositions for various uses which are produced from sustainable chemical feedstocks.

Detergent compositions for household use typically comprise mixtures of surfactants. Commonly used are sulfate surfactants which are high foaming and have excellent detergent properties. However sulfate containing surfactants can be harsh to the skin and it is desirable to minimise skin and ocular irritations as much as possible.

Consumers are therefore seeking detergent compositions that contain more sustainable components and milder, sulfate-free ingredients.

Many sulfate free laundry detergents commonly comprise linear alkyl benzene sulfonate active ingredients. These compounds have been preferred surfactants for such applications due to their good cleaning performance at low temperatures and relatively low cost. However, linear alkyl benzene sulfonates are synthetic anionic surfactants produced from petrochemical feedstocks and are not currently obtainable from renewable sources. They also present further environmental concerns due to potential toxicity to aquatic life. Therefore it is desirable to minimise the levels of such surfactants present in detergent compositions.

Alkoxylated surfactants, such as alcohol ethoxylates, are also commonly present in detergent compositions and provide good foaming properties.

However the undesirable by-product 1,4-dioxane is found in some materials that use ethylene oxide in their production. Due to health and environmental concerns, governments and regulatory bodies are working to reduce the level of 1,4-dioxane in wastewater and have introduced limits on the amount of 1,4-dioxane allowed in consumer products, including household cleaners. There is thus an increased demand for 1,4-dioxane free surfactants.

It is an aim of the present invention to provide a novel detergent composition which overcomes at least one disadvantage of the prior art whether stated herein or otherwise.

In particular the invention seeks to provide detergent compositions which maintain cleaning performance whilst minimising the levels of undesirable ingredients such as sulfates, ethoxylated surfactants and linear and/or branched alkylbenzene sulfonate surfactants.

According to a first aspect of the present invention there is provided a detergent composition comprising:
(a) at least one acyl alkyl isethionate surfactant of formula (I):

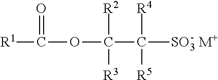

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation; and
   (b) at least one hydrocarbyl saccharide compound.

Component (a) of the detergent composition of the present invention comprises at least one acyl alkyl isethionate of formula (I).

In the formula (I), R¹ represents an optionally substituted C₄-C₃₆ hydrocarbyl group, R², R³, R⁴ and R⁵ each independently represents hydrogen or a substituted or unsubstituted C₁-C₄ alkyl group, provided that at least one of R², R³, R⁴ and R⁵ is not hydrogen, and M⁺ represents a cation.

Suitably, R¹ represents an optionally substituted C₄-C₃₆ alkyl, C₄-C₃₆ alkenyl, C₆-C₁₂ aryl or C₈-C₂₂ alkyl-C₆-C₁₂ aryl group. More suitably, R¹ represents an optionally substituted C₄-C₃₆ alkyl or C₄-C₃₆ alkenyl group, especially an optionally substituted C₄-C₃₆ alkyl group. Most suitably, R¹ represents a C₄-C₃₆ alkyl or C₄-C₃₆ alkenyl group, especially a C₄-C₃₆ alkyl group.

Suitably, R¹ represents an optionally substituted C₄-C₃₆ alkyl or C₄-C₃₆ alkenyl group, such as an optionally substituted C₈-C₁₈ alkyl or C₈-C₁₈ alkenyl group.

Suitably, R¹ represents a C₄-C₃₆ alkyl or C₄-C₃₆ alkenyl group, such as a C₈-C₁₈ alkyl or C₈-C₁₈ alkenyl group.

Suitably, R¹ represents an optionally substituted C₅-C₃₀ alkyl group, such as an optionally substituted C₇-C₂₄ alkyl group, for example an optionally substituted C₇-C₂₁ alkyl group, preferably an optionally substituted C₇-C₁₇ alkyl group.

Suitably, R¹ represents a C₅-C₃₀ alkyl group, such as a C₇-C₂₄ alkyl group, for example a C₇-C₂₁ alkyl group, preferably a C₇-C₁₇ alkyl group.

R¹ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, Ca caprylic acid, C₁₀ capric acid and C₁₈ stearic and oleic acid.

R¹ may include the residue of one or more naturally occurring fatty acids and/or of one or more synthetic fatty acids. For example, R¹ may consist essentially of the residue of a single fatty acid.

Examples of carboxylic acids from which R¹ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from rice bran oil, oat oil, wheat germ oil, hemp seed oil, coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R¹ comprises the residue of coco acid, the residue of mixed fatty acids derived from coconut oil or the residue of mixed fatty acids derived from palm kernel oil. More suitably, R¹ predominantly comprises the residue of a saturated fatty acid having 12 carbon atoms.

By predominantly comprising the residue of a saturated fatty acid having 12 carbon atoms it is meant that at least half of the fatty acid residues that provide R¹ are saturated fatty acids having 12 carbon atoms.

The acyl alkyl isethionate surfactant of the formula (I) may be prepared by any of the methods disclosed in the prior art, for example see the methods described in WO94/09763 and WO2005/075623.

When any of R², R³, R⁴ and R⁵ represents an optionally substituted C₁-C₄ alkyl group, the alkyl group is suitably n-butyl, n-propyl, ethyl or methyl, such as ethyl or methyl, most preferably methyl.

Preferably one of the groups R², R³, R⁴ and R⁵ represents an optionally substituted C₁-C₄ alkyl group and the remaining groups represent hydrogen. For example, R² may represent an optionally substituted C₁-C₄ alkyl group and R³, R⁴ and R⁵ may all represent hydrogen. For example, R⁴ may represent an optionally substituted C₁-C₄ alkyl group and R², R³ and R⁵ may all represent hydrogen.

Preferably, R² represents a C₁-C₄ alkyl group and R³, R⁴ and R⁵ all represent hydrogen. Preferably, R⁴ represents a C₁-C₄ alkyl group and R², R³ and R⁵ all represent hydrogen.

Most preferably, R² represents a methyl group and R³, R⁴ and R⁵ all represent hydrogen. Most preferably, R⁴ represents a methyl group and R², R³ and R⁵ all represent hydrogen.

Suitably, M⁺ represents a metal cation or an optionally substituted ammonium cation, preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable metal cations include alkali metal cations, for example sodium, lithium and potassium cations, and alkaline earth metal cations, for example calcium and magnesium cations. Suitably, M⁺ represents an alkali metal cation or an optionally substituted ammonium cation. Preferably, M⁺ represents a zinc, potassium or sodium cation. Most preferably, M⁺ represents a sodium cation.

The skilled person will appreciate that when M⁺ is a divalent metal cation two moles of anion will be present for each mole of cation.

The acyl alkyl isethionate surfactant of formula (I) may comprise the reaction product of sodium methyl isethionate and a fatty acid, that is a compound of formula R¹COOCHR²CHR⁴SO₃⁻M⁺ in which one of R² and R⁴ is methyl and the other is hydrogen. Mixtures of these isomers may be present.

The detergent composition of the present invention may include a mixture of more than one acyl alkyl isethionate surfactant of formula (I). For example, an isomeric mixture of acyl alkyl isethionate surfactants of formula (I) may be present. Such a mixture may include, for example an acyl alkyl isethionate surfactant in which R² represents a C₁-C₄ alkyl group (suitably methyl) and R³, R⁴ and R⁵ are all hydrogen and an acyl alkyl isethionate surfactant in which R⁴ represents a C₁-C₄ alkyl group (suitably methyl) and R², R³ and R⁵ are all hydrogen.

In particular, the detergent composition of the present invention may comprise a mixture of isomers, that is a compound of formula R¹COOCH₂CHR⁴SO₃⁻M⁺ in which R⁴ represents a C₁-C₄ alkyl group (preferably methyl) and a compound of formula R¹COOCHR²CH₂SO₃⁻M⁺ in which R² represents a C₁-C₄ alkyl group (preferably methyl).

The detergent composition of the present invention may comprise a mixture of isomers in which the molar ratio of the compounds in which R² is C₁-C₄ alkyl and R⁴ is hydrogen to compounds in which R² is hydrogen and R⁴ is C₁-C₄ alkyl is from 99:1 to 1:99, from 99:1 to 1:1, from 98:2 to 2:1, or from 95:5 to 3:1.

Suitably such mixtures comprise approximately 90 mol % of compounds in which R² is C₁-C₄ alkyl and R⁴ is hydrogen and approximately 10 mol % of compounds in which R² is hydrogen and R⁴ is C₁-C₄ alkyl.

Preferably such mixtures comprise approximately 90 mol % of compounds in which R² is methyl and R⁴ is hydrogen and approximately 10 mol % of compounds in which R² is hydrogen and R⁴ is methyl.

Preferably component (a) comprises an acyl alkyl isethionate surfactant of formula (I) selected from one or more of sodium lauroyl methyl isethionate, sodium cocoyl methyl isethionate and sodium oleoyl methyl isethionate. Sodium lauroyl methyl isethionate (SLMI) is especially preferred.

The detergent composition of the first aspect further comprises (b) at least one hydrocarbyl saccharide compound.

By hydrocarbyl saccharide compound we mean to refer to a compound including a hydrocarbyl group and a saccharide moiety.

The hydrocarbyl group may be bound to the saccharide moiety via a carbon-carbon bond or via a carbon-oxygen bond. Preferably it is bound to the saccharide moiety via a carbon-oxygen bond, for example via an ester linkage or an ether linkage. Most preferably it is bound to the saccharide moiety via an ether linkage.

In some embodiments the hydrocarbyl group may be bound to the saccharide moiety via a number of atoms comprising a functional group. In some embodiments the hydrocarbyl saccharide compound may comprise a plurality of hydrocarbyl groups. The saccharide moiety may be bound to the one or more hydrocarbyl groups by one or more bonds.

Preferably the or each hydrocarbyl saccharide compound is a non-ionic surfactant compound

The hydrocarbyl saccharide compound may include one or more hydrocarbyl groups. Preferably it comprises one hydrocarbyl group. The hydrocarbyl group may be an optionally substituted alkyl, alkenyl, alkynyl, aryl, alkaryl or aralkyl group. Preferably the hydrocarbyl group is an unsubstituted hydrocarbyl group. It may be straight chained or may be branched. Most preferably it is straight chained. Preferred hydrocarbyl groups are alkyl, alkenyl, alkynyl, aryl, alkaryl or aralkyl groups having from 1 to 36 carbon atoms, preferably 2 to 30 carbon atoms, more preferably from 4 to 24 carbon atoms, suitably from 4 to 20 carbon atoms, preferably from 6 to 16 carbon atoms, for example from 8 to 16 carbon atoms and most preferably from 10 to 14 carbon atoms. Preferred are straight chained alkyl or alkenyl groups having from 6 to 16 carbon atoms.

Especially preferred hydrocarbyl groups are alkyl groups having from 1 to 36 carbon atoms, preferably 2 to 30 carbon atoms, more preferably from 4 to 24 carbon atoms, suitably from 4 to 20 carbon atoms, preferably from 6 to 16 carbon atoms, for example from 8 to 16 carbon atoms and most preferably from 10 to 14 carbon atoms. Preferred are straight chained alkyl groups having from 6 to 16 carbon atoms.

The saccharide moiety of the hydrocarbyl saccharide compound may include monosaccharide, disaccharide, oligosaccharide or polysaccharide species.

The saccharide moiety of the hydrocarbyl saccharide compound may include from 1 to 30 monosaccharide species. Preferably the saccharide moiety comprises from 1 to 10, suitably from 1 to 5, preferably from 1 to 2 monosaccharide units. Any suitable monosaccharide unit may be included. Preferred monosaccharides include allose, altrose, glucose, mannose, gulose, idose, galactose, rhamnose and talose.

In some embodiments the saccharide moiety comprises rhamnose or sophorose.

In some embodiments the hydrocarbyl saccharide compound may be a glycolipid biosurfactant. Such surfactant compounds will be known to the person skilled in the art and can be obtained by microbial fermentation. In some such embodiments the saccharide moiety comprises 1 to 2 rhamnose units and in some such embodiments the saccharide moiety comprises 1 sophorose unit. Suitable glycolipid biosurfactants include rhamnolipids, sophorolipids, mannosylerythritol lipids, trehalolipids, cellobioselipids and derivatives and/or mixtures thereof.

Mixtures of two or more monosaccharides may be present in the saccharide moiety. Preferably the saccharide moiety comprises glucose. More preferably all of the monosaccharide units present in the saccharide moiety are glucose.

In a preferred embodiment the hydrocarbyl saccharide compound is an alkyl saccharide compound, preferably an alkyl glucoside (APG), more preferably a monoalkyl-glucoside. Suitably the hydrocarbyl-saccharide compound is a compound of general formula (II):

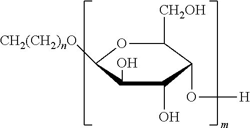

wherein n is from 2 to 24, preferably from 5 to 17, more preferably from 9 to 13 and m is from 1 to 10, preferably from 1 to 2.

Preferably, on average, the hydrocarbyl saccharide compound includes 1 to 1.5, for example about 1.3 glucose units per molecule. As the skilled person will appreciate, a single molecule cannot have 1.3 glucose units. What is meant by an average number of glucose units is the mean number of glucose units per alkyl chain as the sum of all the glucose units present in a sample divided by the total number of molecules of hydrocarbyl saccharide compound.

Component (b) may comprise an alkyl glucoside surfactant.

Component (b) may comprise glycolipid biosurfactant.

In some embodiments component (b) comprises an alkyl glucoside surfactant and a glycolipid biosurfactant.

In preferred embodiments component (b) comprises an alkyl glucoside, preferably lauryl glucoside and/or decyl glucoside.

In some embodiments the detergent composition of the first aspect of the present invention further comprises (c) a taurate surfactant.

Component (c) may comprise an alkyl acyl taurate of formula (III):

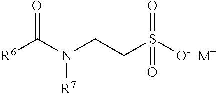

in which R⁶ is a C₅ to C₃₀ alkyl group, such as a C₁₀ to C₂₀ alkyl group and R⁷ is a C₁ to C₆ alkyl group, such as a C₁ to C₄ alkyl group.

Each of R⁶ and R⁷ may be an unsubstituted alkyl group.

R⁶ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, Ca caprylic acid, C₁₀ capric acid and C₁₈ stearic and oleic acid.

R⁶ may include the residue of one or more naturally occurring fatty acids and/or of one or more synthetic fatty acids. For example, R⁶ may consist essentially of the residue of a single fatty acid.

Examples of carboxylic acids from which R⁶ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from rice bran oil, oat oil, wheat germ oil, hemp seed oil, coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof.

Preferably the compound of formula (III) comprises a methyl acyl taurate. Examples of suitable alkyl acyl taurates include sodium lauroyl methyl taurate, sodium methyl myristoyl taurate, sodium methyl palmitoyl taurate, sodium methyl stearoyl taurate, sodium methyl cocoyl taurate, sodium methyl oleoyl taurate, and combinations thereof.

Preferably component (c) comprises sodium methyl oleoyl taurate, sodium methyl cocoyl taurate or mixtures thereof.

Alternatively and/or additionally component (c) may comprise an alkyl acyl taurate of formula (IV):

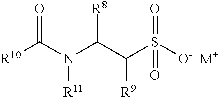

wherein:
R⁸ and R⁹ are each independently selected from H or C₁₄ alkyl, provided that one of R⁸ and
R⁹ is H and the other of R⁸ and R⁹ is C₁₋₄ alkyl;
R¹¹ is C₁₋₆ alkyl, C₂₋₆ alkenyl, or C₁₋₆ alkyl substituted with an aryl group; and
R¹⁰ is C₄₋₂₅ alkyl or C₄₋₂₅ alkenyl wherein the C₄₋₂₅ alkyl or C₄₋₂₅ alkenyl is optionally substituted by hydroxy.

Preferably one of R⁸ and R⁹ is C₁₋₄ alkyl and the other is H. More preferably one of R⁸ and R⁹ is methyl and the other is H.

Preferably R¹¹ is C₁₋₆ alkyl or C₂₋₆ alkenyl, preferably, C₁₋₆ alkyl, such as C₁₋₂ alkyl. More preferably, R¹¹ is methyl.

R¹⁰ is preferably C₈₋₁₈ alkyl or C₈₋₁₈ alkenyl, wherein the C₈₋₁₈ alkyl or C₈₋₁₈ alkenyl, is optionally substituted by hydroxy. Preferably R¹⁰ is an unsubstituted C₈₋₁₈ alkyl or an unsubstituted C₈₋₁₈ alkenyl.

R¹⁰ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, Ca caprylic acid, C₁₀ capric acid and C₁₈ stearic and oleic acid.

R¹⁰ may include the residue of one or more naturally occurring fatty acids and/or of one or more synthetic fatty acids. For example, R¹⁰ may consist essentially of the residue of a single fatty acid.

Examples of carboxylic acids from which R¹⁰ may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from rice bran oil, oat oil, wheat germ oil, hemp seed oil, coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof.

Preferably R¹⁰ is an unsubstituted C₄₋₂₅ alkyl, such as an unsubstituted C₈₋₁₈ alkyl.

The detergent composition of the first aspect of the present invention comprises component (a), component (b), and optionally component (c).

The weight ratio of component (a) to component (b) is preferably from 100:1 to 1:50, suitably from 50:1 to 1:10, preferably from 10:1 to 1:5, more preferably from 5:1 to 1:1, for example from 2:1 to 1:3.

The weight ratio of component (a) to component (c), when present, is preferably from 50:1 to 1:100, preferably from 10:1 to 1:30, more preferably from 2:1 to 1:5.

The weight ratio of component (b) to component (c), when present is preferably from 50:1 to 1:600, preferably from 5:1 to 1:10, more preferably from 1:1 to 1:3.

The above amounts refer to the weight ratios of the active surfactant compound present in the detergent composition and do not include any residual starting materials, impurities or by products that may be present.

In embodiments in which component (a) and/or component (b) and/or component (c) comprises a mixture of compounds, these ratios are based on the total amount of each such component present in the composition.

In addition to components (a), (b) and optionally (c), the detergent compositions of the present invention may optionally comprise one or more additional surfactants.

Such surfactants may be selected from anionic surfactants, cationic surfactants, non-ionic surfactants, amphoteric or zwitterionic surfactants and mixtures thereof. The selection of suitable further surfactants for use in the composition of the present invention is within the competence of the person skilled in the art.

In some embodiments the detergent composition further comprises (d) one or more amphoteric or zwitterionic surfactants.

Suitable amphoteric surfactants for use in the detergent compositions of the present invention include those based on fatty nitrogen derivates and those based on betaines.

Suitable amphoteric or zwitterionic surfactants may be selected from betaines, for example alkyl betaines, alkylamidopropyl betaines, for example cocamidopropyl betaine, alkylamidopropyl hydroxy sultaines, alkylamphoacetates, alkylamphodiacetates, alkyl propionates, alkylamphodipropionates, alkylamphopropionates, alkyliminodipropionates and alkyliminodiacetate.

Amphoteric or zwitterionic surfactants for use in compositions of the first aspect may include those which have an alkyl or alkenyl group of 7 to 22 carbon atoms and comply with an overall structural formula:

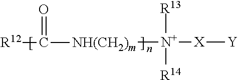

where R¹² is alkyl or alkenyl of 7 to 22 carbon atoms; R¹³ and R¹⁴ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂⁻ or —SO₃⁻.

Amphoteric or zwitterionic surfactants may include simple betaines of formula:

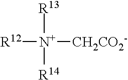

and amido betaines of formula:

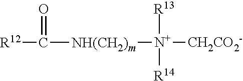

where m is 2 or 3.
In both formulae R¹², R¹³ and R¹⁴ are as defined previously. R¹² may, in particular, be a mixture of C₁₂ and C₁₄ alkyl groups derived from coconut so that at least half, preferably at least three quarters, of the groups R¹² has 10 to 14 carbon atoms. R¹³ and R¹⁴ are preferably methyl.

Amphoteric or zwitterionic surfactants may include sulfobetaines of formula:

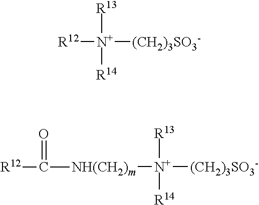

where m is 2 or 3, or variants of these in which
—(CH₂)₃SO₃⁻ is replaced by

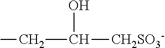

where R¹², R¹³ and R¹⁴ in these formulae are as defined previously.

Amphoteric or zwitterionic surfactants may include amphoacetates and diamphoacetates. Amphoacetates generally conform to the following formula:

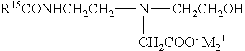

Diamphoacetates generally conform to the following formula:

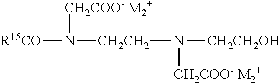

where R¹⁵ is an aliphatic group of 8 to 22 carbon atoms and M₂⁺ is a cation such as sodium, potassium, ammonium, or substituted ammonium.

Suitable acetate-based surfactants include lauroamphoacetate; alkyl amphoacetate; sodium alkyl amphoacetate; cocoampho (di)acetate; cocoamphoacetate; disodium cocoamphodiacetate; sodium cocoamphoacetate; disodium capryloamphodiacete; disodium lauroamphoacetate; sodium lauroamphoacetate and disodium wheat germamphodiacetate.

Suitable betaine surfactants include alkylamido betaine; alkyl betaine, C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, C₈ amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow aminobetaine.

Suitable glycinate surfactants include acyl glycinates such as cocoamphocarboxyglycinate; tallowamphocarboxyglycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C_{10/12} fatty acid amidoethyl-N-(2-hydroxyethyl)-glycinate; N—C_{12/18}-fatty acid amidoethyl-N-(2-hydroxyethyl)-glycinate; dihydroxyethyl tallow gycinate, sodium lauroyl glycinate and sodium cocoyl glycinate.

Preferred acetate-based amphoteric surfactants for use herein include sodium lauroamphoacetate, disodium lauroamphoacetate and mixtures thereof.

Preferred betaine surfactants for use as herein include cocoamidopropyl betaine.

Preferred sultaine surfactants for use as herein include cocoamidopropylhydroxy sultaine.

In preferred embodiments component (d) comprises a betaine surfactant, preferably an alkyl amido betaine surfactant.

Most preferably component (d) comprises cocoamidopropyl betaine (CAPB).

In some embodiments the detergent composition of the present invention may comprise one or more additional surfactants, for example one or more additional anionic surfactants.

Suitable anionic surfactants for use in compositions of the first aspect of the invention include salts of C₁₂-C₁₈ carboxylic acids, ethoxylated carboxylic acids, ester carboxylates and ethoxylated ester carboxylates and sarcosinates.

Suitable anionic surfactants may be selected from salts of fatty acids; alkali metal salts of mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, non-acylated alkyl isethionates; fatty acid taurates; acyl taurates; amino acid surfactants such as glutamates and glycinates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids and salts of lipoamino acids. Particularly exemplary salts of the above, where applicable, are the sodium, potassium, ammonium, magnesium and triethanolamine salts. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Preferred anionic surfactants do not include sulfates or linear and/or branched alkylbenzene sulfonates.

Preferred anionic surfactants are selected from salts of fatty acids; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, non-acylated alkyl isethionates; fatty acid taurates; acyl taurates; amino acid surfactants such as glutamates and glycinates; products of condensation of fatty acids with oxy- and aminoalkanesulfonic acids; alkyl and acyl sarcosinates; sulfoacetates; alkyl phosphates; alkyl phosphate esters; acyl lactates; and salts of lipoamino acids. Particularly exemplary salts of the above, where applicable, are the sodium, potassium, ammonium, magnesium and triethanolamine salts. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable sulfoacetates include acyl sulfoacetates, particularly sodium acyl sulfoacetates.

Suitable glutamate surfactants include acyl glutamates.

Suitable acyl isethionate surfactants are compounds of formula R¹⁶COOCH₂CH₂SO₃⁻M⁺ wherein R¹⁶ represents an optionally substituted C₄-C₃₆ hydrocarbyl group; and M⁺ represents a cation.

Suitably R¹⁶ represents an optionally substituted C₄-C₃₆ alkyl, C₄-C₃₆ alkenyl, C₆-C₁₂ aryl or C₈-C₂₂ alkyl-C₆-C₁₂ aryl group.

Preferably R¹⁶ represents an optionally substituted C₅-C₃₀ alkyl group, preferably a C₇-C₂₄ alkyl group, for example a C₇-C₂₁ alkyl group, preferably a C₇-C₁₇ alkyl group.

R¹⁶ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, Ca caprylic acid, and C₁₈ stearic and oleic acid.

R¹⁶ may include the residue of one or more naturally occurring fatty acids and/or of one or more synthetic fatty acids.

Suitably, M⁺ represents a metal cation or an optionally substituted ammonium cation, preferably a metal cation. Suitable ammonium cations include NH₄⁺ and those derived from alkyl amines and alkanolamines, for example the ammonium cation of triethanolamine.

Suitable metal cations include alkali metal cations and alkaline earth metal cations. Preferably M⁺ represents a zinc, potassium or sodium cation. Most preferably M⁺ represents a sodium cation.

The skilled person will appreciate that when M⁺ is a divalent metal cation two moles of anion will be present for each mole of cation.

In some embodiments only a single acyl isethionate may be present in the detergent composition. In some embodiments a mixture of two or more acyl isethionates of the formula (II) may be present.

Preferred acyl isethionates include sodium lauroyl isethionate, sodium cocoyl isethionate and sodium myristoyl isethionate. Sodium cocoyl isethionate is especially preferred.

In some embodiments the detergent composition of the first aspect may optionally comprise one or more non-ionic surfactants. Such non-ionic surfactants are present in addition to the hydrocarbyl saccharide compound or compounds present in component (b).

Suitable non-ionic surfactants for use in compositions of the first aspect of the invention include alcohol alkoxylates such as alcohol ethoxylates, alcohol propoxylates, and ethylene oxide/propylene oxide copolymer derived surfactants, aliphatic esters, aromatic esters, sugar esters, especially sorbitan esters, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides and amine oxides.

Preferred non-ionic surfactants for use herein are compounds which are not ethoxylated.

In some embodiments the detergent composition of the first aspect may optionally comprise one or more cationic surfactants.

Suitable cationic surfactants for use in compositions of the first aspect of the invention are typically based on fatty amine derivates or phosphonium quaternary ions, and quaternary ammonium compounds. Polymeric cationic surfactancts may also be used.

Suitable cationic surfactants for use in compositions of the first aspect of the invention include tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate.

Examples of suitable cationic surfactants include quaternary ammonium compounds, particularly trimethyl quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen (e.g. bromide), acetate, citrate, lactate, glycolate, phosphate nitrate, sulfate, or alkylsulfate.

Further suitable cationic surfactants include those materials having the CTFA designations Quaternium-5, Quaternium-31 and Quaternium-18. Mixtures of any of the foregoing materials may also be suitable.

Salts of primary, secondary, and tertiary fatty amines are also suitable cationic surfactants. The alkyl groups of such amines preferably have from 12 to 22 carbon atoms, and can be optionally substituted.

Useful cationic surfactants include amido substituted tertiary fatty amines, in particular tertiary amines having one C12 to C22 alkyl or alkenyl chain. Such amines include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachid amidopropyldiethylamine, arachidamidoethyldiethylamine, arachidamidoethyldimethylamine, diethylaminoethylstearamide.

Also useful are dimethylstearamine, dimethylsoyamine, soyamine, myristylamine, tridecylamine, ethylstearylamine, N-tallow propanediamine, ethoxylated (with 5 moles of ethylene oxide) stearylamine, dihydroxyethylstearylamine, and arachidyl behenylamine.

These amines are typically used in combination with an acid to provide the cationic species. Suitable acids include L-glutamic acid, lactic acid, hydrochloric acid, sulfuric acid, nitric acid, phosphoric acid, malic acid, succinic acid, acetic acid, fumaric acid, tartaric acid, citric acid, L-glutamic hydrochloride, and mixtures thereof; more preferably L-glutamic acid, lactic acid, citric acid.

Other useful cationic amine surfactants include those disclosed in U.S. Pat. No. 4,275,055.

Suitable polymeric cationic surfactants include polyquaternium-7, polyquaternium-10, polyquaternium-11, guar hydroxypropyltrimonium chloride, and hydroxypropyl guar hydroxypropyltrimonium chloride.

The detergent composition of the first aspect comprises a mixture of surfactants.

In some embodiments the detergent composition comprises component (a), component (b) and component (c).

In some embodiments the detergent composition comprises component (a), component (b) and component (d).

Preferably component (a), component (b) and, when present, component (c) and/or component (d) together make up at least 50 wt % of all surfactant compounds present in the composition, preferably at 60 wt %, more preferably at least 70 wt %. Preferably component (a), component (b) and, when present, component (c) and/or component (d) together make up at least 80 wt % of all surfactant compounds present in the composition, preferably at least 85%, for example at least 90 wt % or at least 95 wt %.

In some embodiments the detergent composition of the first aspect of the present invention may be a liquid or solid precursor composition.

In such embodiments the detergent composition could be used as a multi-component ingredient for inclusion in formulated compositions.

However in preferred embodiments the detergent composition of the present invention is a formulated detergent composition intended to be used for a particular purpose.

The present invention may provide a detergent composition for use in toilet care, automatic dishwashing, manual dishwashing, laundry, fabric care, kitchen care, carpet cleaning, air fresheners, vehicle care, polishing products, machine cleaning and maintenance, pesticides, insecticides, fungicides, herbicides, oilfield chemical applications, marine applications, personal care and institutional/industrial cleaning. The detergent compositions may be especially useful in household cleaning applications, for example as laundry detergent composition.

Component (a) is preferably present in the detergent composition of the present invention in an amount of at least 0.5 wt %, preferably at least 1 wt %, suitably at least 2 wt %.

Component (a) is preferably present in the detergent composition of the present invention in an amount of up to 60 wt %, preferably up to 30 wt % suitably up to 15 wt %.

Component (a) may be present in the detergent composition of the present invention in an amount of from 0.5 to 60 wt %, 1 to 30 wt % or 2 to 15 wt %.

Component (b) is preferably present in the detergent composition of the present invention in an amount of at least 0.1 wt %, preferably at least 0.5 wt %, more preferably at least 1 wt %.

Component (b) is preferably present in the detergent composition of the present invention in an amount of up to 30 wt %, preferably up to 20 wt % suitably up to 10 wt %.

Component (b) may be present in the detergent composition of the present invention in an amount of from 0.1 to 30 wt %, 0.5 to 20 wt % or 1 to 10 wt %.

In some embodiments the detergent composition comprises component (c). In such embodiments component (c) is preferably present in the detergent composition of the present invention in an amount of at least 0.5 wt %, preferably at least 1 wt %, more preferably at least 2 wt %.

Component (c) is preferably present in the detergent composition of the present invention in an amount of at least 40 wt %, preferably up to 25 wt %, more preferably up to 15 wt %.

Component (c) may be present in the detergent composition of the present invention in an amount of from 0.5 to 40 wt %, 1 to 25 wt % or 2 to 15 wt %.

In some embodiments the detergent composition comprises component (d). In such embodiments component (d) is preferably present in the detergent composition of the present invention in an amount of at least 0.5 wt %, preferably at least 1 wt %, more preferably at least 2 wt %.

Component (d) is preferably present in the detergent composition of the present invention in an amount of up to 18 wt %, more preferably up to 15 wt %.

Component (d) may be present in the detergent composition of the present invention in an amount of from 0.5 to 18 wt %, 1 to 17 wt % or 2 to 15 wt %.

The above amounts refer to the weight ratios of the active surfactant compound present in the detergent composition and do not include any residual starting materials, impurities or by products that may be present.

In some preferred embodiments the detergent composition comprises from 1 to 30 wt %, preferably 10 to 20 wt % component (a) and 0.1 to 10 wt %, preferably 0.5 to 5 wt % component (b).

In some preferred embodiments the detergent composition comprises from 0.1 to 20 wt %, preferably 1 to 10 wt % component (a); 0.1 to 10 wt %, preferably 0.5 to 5 wt % component (b) and 0.1 to 20 wt %, preferably 1 to 10 wt % component (c).

In some preferred embodiments the detergent composition comprises from 0.1 to 20 wt %, preferably 2 to 15 wt % component (a); 0.1 to 10 wt %, preferably 0.5 to 5 wt % component (b) and 0.1 to 18 wt %, preferably 2 to 15 wt % component (d).

Preferably component (a), component (b), and, when present, component (c) and/or component (d) together provide less than 30 wt % of the detergent composition of the first aspect, preferably less than 25 wt %, suitably less than 22 wt %, preferably less than 20 wt %. Thus the total amount of components (a), (b), (c) and (d) present in the detergent composition of the first aspect is preferably less than 30 wt %, suitably less than 25 wt %, preferably less than 22 wt %, for example less than 20 wt %.

The detergent composition of the first aspect of the present invention preferably comprises less than 30 wt % surfactant compounds in total, based on the total weight of the composition, preferably less than 28 wt %, suitably less than 25 wt %, for example less than 22 wt % or less than 20 wt %.

For the avoidance of doubt the above amounts refer to the active amount of each component ignoring any residual starting material, byproduct or impurity that may be present.

The combination of surfactants used in the detergent compositions of the present invention provides excellent cleaning performance and the compositions do not require the inclusion of alkoxylated compounds which could lead to the presence of 1,4-dioxane; or sulfates which can lead to skin and ocular irritation; or linear and/or branched alkylbenzene sulfonates which are derived from petrochemical sources.

In preferred embodiments the detergent composition of the present invention comprises very low levels or no 1,4-dioxane.

Preferably the detergent composition of the present invention comprises less than 1000 ppm 1,4-dioxane, preferably less than 500 ppm, more preferably less than 250 ppm or less than 100 ppm, for example less than 50 ppm, less than 20 ppm or less than 10 ppm. Preferably the detergent composition of the present invention is substantially free of 1,4-dioxane.

In this specification ppm refers to parts per million by weight, unless otherwise specified.

Suitably the detergent compositions of the present invention comprise low levels of alkoxylated, especially ethoxylated surfactants.

In preferred embodiments the detergent composition of the first aspect comprises less than 2.5 wt % alkoxylated surfactants, preferably less than 2 wt %, more preferably less than 1.5 wt %, preferably less than 1 wt %, suitably less than 0.75 wt %, more preferably less than 0.5 wt %, preferably less than 0.25 wt %, preferably less than 0.1 wt %, suitably less than 0.05 wt %, for example less than 0.01 wt %, preferably less than 0.005 wt % and most preferably less than 0.001 wt %.

In preferred embodiments the detergent composition of the first aspect comprises less than 2.5 wt % ethoxylated surfactants, preferably less than 2 wt %, more preferably less than 1.5 wt %, preferably less than 1 wt %, suitably less than 0.75 wt %, more preferably less than 0.5 wt %, preferably less than 0.25 wt %, preferably less than 0.1 wt %, suitably less than 0.05 wt %, for example less than 0.01 wt %, preferably less than 0.005 wt % and most preferably less than 0.001 wt %.

By alkoxylated or ethoxylated surfactants we mean to refer to any surfactant compound which includes an alkoxy or ethoxy repeat unit.

Preferably the detergent composition of the present invention is substantially free of alkoxylated surfactants.

Preferably the detergent composition of the present invention is substantially free of ethoxylated surfactants.

In some preferred embodiments the detergent composition of the present invention comprises less than 2.5 wt % sulfate containing surfactants, preferably less than 2 wt %, more preferably less than 1.5 wt %, preferably less than 1 wt %, suitably less than 0.75 wt %, more preferably less than 0.5 wt %, preferably less than 0.25 wt %, preferably less than 0.1 wt %, suitably less than 0.05 wt %, for example less than 0.01 wt %, preferably less than 0.005 wt % and most preferably less than 0.001 wt %.

In some embodiments the detergent composition of the first aspect of the present invention is preferably free of sulfate containing surfactants.

Suitably the detergent composition comprises less than 10 wt %, less than 5 wt % or less than 1 wt % of alkylbenzenesulfonates (linear and branched). Specifically, the detergent composition preferably comprises less than 10 wt %, less than 5 wt % or less than 1 wt % of linear alkylbenzenesulfonates and preferably no branched alkylbenzenesulfonates. Preferably the detergent composition is substantially free of alkylbenzenesulfonates. Suitably the detergent composition comprises no linear alkylbenzenesulfonates and no branched alkylbenzenesulfonates.

The detergent composition of the present invention may comprise one or more further components. Suitable further components include chelating agents, preservatives, pH adjusting agents and hydrotropes.

The detergent composition of the present invention may comprise a chelating agent. Suitable chelating agents include ethylenediamine-N,N′-disuccinic acid, methylglycinediacetic acid, glutamic acid N,N-diacetic acid, imino disuccinic acid, diethylene triamine pentaacetic acid, ethylenediamine tetraacetic acid, diethylenetriamine penta methylene phosphonic acid, etidronic acid, nitrilotriacetic acid and anions, salts and mixtures thereof.

Preferred chelating agents are biodegradable chelating agents for example ethylenediamine-N,N′-disuccinic acid, methylglycinediacetic acid, glutamic acid N,N-diacetic acid, imino disuccinic acid and anions and mixtures thereof. Ethylenediamine-N,N′-disuccinic acid (EDDS) is especially preferred. The skilled person will appreciate that polycarboxylic acid chelating agents may be present as the free acid or a salt thereof.

The detergent composition of the present invention may comprise a preservative. Suitable preservatives will be known to the person skilled in the art and include sodium benzoate, potassium sorbate, sorbic acid, phenoxyethanol, benzyl alcohol, DMDM hydantoin, imidazolidinyl urea, methylchloroisothiazolinone, methylisothiazolinone, salicylic acid, benzyl salicylate, methylparaben, propylparaben and caprylyl glycol. A preferred preservative for use herein is sodium benzoate.

The detergent composition of the present invention may comprise a hydrotrope. Suitable hydrotropes will be known to the person skilled in the art and include propylene glycol, hexylene glycol, glycerine, sorbitol, xylene sulfonates, cumene sulfonates, ethanol, urea, dipropylene glycol. A preferred hydrotrope for use herein is sorbitol.

The detergent composition of the present invention may comprise a pH adjusting agent. Suitable pH adjusting agents will be known to the person skilled in the art and include citric acid, lactic acid, potassium hydroxide, sodium hydroxide, sodium carbonate, triethanolamine and sodium gluconate.

Preferably the detergent composition of the present invention has a pH of from 4.5 to 11, preferably 5 to 9, for example 7 to 8.5.

The detergent composition of the present invention may be a solid or liquid form. In some embodiments the composition is provided as a solid block. In some embodiments the composition comprises compressed powder. In some embodiments the composition comprises a free flowing particulate material.

The detergent composition of the first aspect may be a liquid composition or semi-liquid composition. It may be a thick viscous liquid in the form of a thick paste or it may be a runny liquid.

Preferably liquid compositions of the present invention (especially laundry detergent compositions) have a viscosity at least 100 cP, preferably at least 500 cP, more preferably at least 1000 cP or at least 2000 cP. Suitably liquid (especially laundry) detergent compositions of the invention have a viscosity in the range 2500 to 100000 cP, preferably 3000 to 70000 cP.

Viscosities may suitably be measured with a Brookfield Viscosimeter at 25° C., Spindle 21-29, 10-50 RPM.

The detergent composition of the present invention when in liquid form, is preferably an aqueous composition. In some embodiments the composition may comprise one or more further solvents in addition to water. Such suitable co-solvents will be known to the person skilled in the art.

However in preferred embodiments water is the major solvent present in the detergent composition of the present invention and suitably comprises at least 80 wt % of all solvents present, preferably at least 90 wt %, more preferably at least 95 wt %.

Preferably the detergent composition is an isotropic composition. Preferably the detergent composition does not include a lamellar phase.

In some preferred embodiments the detergent composition of the first aspect comprises component (a), component (b) and component (c) and is in the form of a solid.

In some preferred embodiments the detergent composition of the first aspect comprises component (a), component (b) and component (d) and is in the form of a liquid.

Other additional components that may be present in the detergent compositions of the present invention will depend on the intended use of the composition.

For example laundry and dishwashing compositions typically comprise ingredients such as surfactants, fillers, builders, bleaches, bleach activators, redeposition additives, dye transfer inhibitors, enzymes, colorants and fragrances.

Personal care compositions typically comprise ingredients such as surfactants (including anionic, amphoteric, nonionic and cationic surfactants); conditioning agents (including quaternary ammonium compounds, cationic polymers, cationic conditioning polymers, silicones, synthetic or natural oils or resins etc), fatty alcohols, electrolytes or other rheology modifiers, opacifying/pearlising agents, scalp benefit agents, fragrances, dyes, UV filters, penetration enhancers (eg, propylene carbonate, benzyl alcohol etc), preservatives, antioxidants, emulsifiers, pH adjusting agents and buffers and styling polymers (eg, polyvinylpyrrolidone etc).

The detergent composition of this may further comprise one or more solvents. In some embodiments the composition comprises one or more solvents including at least one hydroxy functional group. Suitable such solvents include monohydric alcohols, polyhydric alcohols and alkoxy alcohols. Preferred solvents are miscible with water.

Suitable simple monohydric alcohols include methanol, ethanol, isopropanol and butanol.

In some embodiments the composition comprises a polyhydric alcohol or an alkoxy alcohol.

Suitable alkoxy alcohols include diethylene glycol monobutyl ether, 3-methoxy-3-methyl-1-butanol and 2-butoxyethanol.

One especially preferred alkoxy alcohol for use herein is 3-methoxy-3-methyl-1-butanol.

Suitable polyhydric alcohols include glycerol, ethylene glycol, polyethylene glycol, propylene glycol, dipropylene glycol and polypropylene glycol. Preferred polyhydric alcohols are propylene glycol or a mixture of propylene glycol and glycerol.

In some embodiments, the detergent composition may further comprise a second solvent in addition to the solvent including at least one hydroxy functional group. The second solvent may be a non-water miscible solvent. Suitable such solvents may be selected from one or more of hydrocarbon oils, alkylcarbonates, synthetic and vegetable oils, essential oils, oily esters, silicon oils or perfumes.

In some embodiments the detergent composition may comprise one or more enzymes. Suitable enzymes for use in detergent compositions, for example laundry detergent compositions, are known in the art. The enzyme may be selected from a protease, an amylase, a mannanase or a lipase, or a mixture thereof. The enzyme of may improve the cleaning performance of the detergent composition, in particular in laundry detergent applications, in particular at relatively low washing temperatures.

Suitable additional components for use in automatic dishwashing compositions may include corrosion inhibitors, scents, perfume carriers, chelating agents, bleaching agents, bleach activators, biocides, dyes, pigments, fragrances, pH adjusting agents, builders (preferably silicates or zeolites), peroxide based compounds (especially hydrogen peroxide) chlorine bleaches, perborate compounds, bleach activators and catalysts.

When the detergent composition is a dishwashing composition it may include any of these components.

Preferably the detergent composition is a laundry detergent composition.

Suitable additional components for inclusion in laundry compositions will be known to the person skilled in the art and include chelating agents, fillers, builders, bleaching agents, bleach activators, pigments, dispersants, polymeric dispersing agents, dyes, dye transfer inhibitors, fragrances, fragrance delivery systems, enzyme stabilizers, biocides, probiotics, preservatives, pH adjusting agents, phosphates, silicates, zeolites, peroxide based compounds (especially hydrogen peroxide, chlorine bleaches), perborate compounds, percarbonate compounds, bleach activators and catalysts, cationic surfactants, redeposition additives, brighteners, sud suppressors, fabric softeners and hydrotropes.

The laundry compositions of the present invention preferably comprise one or more builders. Suitable builders will be known to the person skilled in the art and include, for example, sodium sulfate, sodium bicarbonate, sodium carbonate and sodium citrate

In some preferred embodiments the detergent composition of the present invention is a laundry composition, preferably a solid laundry composition comprising from 10 to 95 wt % builders, for example 30 to 90 wt % or from 60 to 85 wt %.

Percarbonate and/or perborate compositions may be present in laundry detergent compositions, especially solid laundry detergent compositions of the invention in an amount of from 1 to 25 wt %, for example 2 to 20 wt %, preferably 5 to 15 wt %.

In some embodiments the composition comprises an anti-redeposition agent. These compounds help maintain salts in solution during the machine wash cycle.

Suitable anti-redeposition agents will be known to the person skilled in the art. Such components often comprise polyimides. One suitable compound is sold under the trade mark Sokalan HP20 and is an ethoxylated polyethylene polyimine.

Anti-redeposition agents are typically included in an amount of from 0.1 to 2 wt %.

Preferably the composition or the first aspect comprises a chelating agent. Suitable chelating agents will be known to the person skilled in the art, and are as previously defined herein.

Some preferred chelating agents for use herein are phosphonate chelating agents.

Chelating agents are typically included in an amount of from 0.1 to 2 wt %.

Other preferred components for inclusion in the detergent compositions of the present invention include probiotics, dye transfer inhibitors and wash performance boosting polymers.

Wash performance boosting polymers are known to those skilled in the art. For example, the detergent composition may comprise polyacrylates, polycarboxylates, cellulose derivatives, polyesters based on terephthalic acid, ethylene/propylene glycol-based polymers, poly(vinylpyrrolidone (PVP), poly(vinylpyridine N-oxide) (PVP-NO), poly(vinylpyridine betaine), biodegradable polymeric dispersants (for example polyamino acid polymers such as polyaspartate and polysaccharides such as oxidized starch), poly(vinyl alcohol), polyalkylene glycol and copolymers of alkylene oxide and vinyl acetate, modified ethoxylated urethane or multifunctional polyethylene imine.

Dye transfer inhibitors are also known to those skilled in the art.

Preferably the compositions of the present invention are sufficiently non-irritating and non-toxic that they can be directly handled by the user.

Suitably the detergent composition is readily dissolvable and/or dispersible in water.

In some embodiments the detergent composition of this first aspect is a liquid laundry detergent composition.

In some preferred embodiments the detergent composition is a solid laundry detergent composition.

The detergent composition may be provided in unit dose form.

In some embodiments, the present invention provides a solid laundry composition in unit dose form.

Preferably each unit dose has a mass of from 5 to 100 g, preferably from 5 to 75 g, more preferably from 10 to 50 g.

In some embodiments the unit dose of the detergent composition may be provided in a water-soluble film or sachet, which dissolves in use on exposure to water to release the detergent composition from the sachet. Suitable water-soluble film sachets or known in the art and may be formed from polyvinyl alcohol.

In some embodiments the present invention provides a unit dose detergent product comprising a portion of a detergent composition according to the first aspect and a wrapper.

Suitably the portion of the detergent composition is an amount suitable for washing a standard load of laundry.

The portion of the detergent composition may be in solid or liquid form, preferably in solid form.

Preferably the present invention provides a solid laundry composition in unit dose form.

The portion of the detergent composition is suitably surrounded by the wrapper. Suitably the detergent composition is sealed within the wrapper.

The wrapper is suitably made from a thin flexible film.

In some embodiments the wrapper may comprise a water soluble film (e.g. of polyvinyl alcohol) intended to be dosed in a washing machine.

In some embodiments the wrapper may comprise a protective polymeric film which is to be removed before dosing into a washing machine. Such films are suitably frangible and made from material which protects the enclosed detergent composition from moisture and air.

In some embodiments the unit dose detergent product may comprise a water soluble wrapper and a protective polymeric film wrapper.

Preferably the compositions of the present invention are sufficiently non-irritating and non-toxic that they can be directly handled by the user. Thus in some preferred embodiments unit doses of the detergent composition of the present invention do not comprise any individual coating, encapsulation or wrapping.

The unit dose forms may be provided in plastic free packaging.

In some embodiments, the unit dose form comprises the detergent composition of the first aspect and a second composition.

In some embodiments pouches or tablets include multiple compartments. This is typically to separate incompatible ingredients so that they do not react and degrade on storage. Such embodiments are within the scope of the invention.

In some preferred embodiments the present invention provides a laundry detergent composition comprising:
(a) at least one acyl alkyl isethionate surfactant of formula (I):

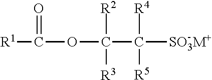

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
   (b) at least one hydrocarbyl saccharide compound; optionally
   (c) a taurate surfactant; and optionally
   (d) one or more amphoteric or zwitterionic surfactants.

In some preferred embodiments the present invention provides a solid laundry detergent composition comprising:
(a) at least one acyl alkyl isethionate surfactant of formula (I):

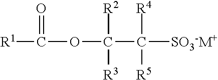

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
   (b) at least one hydrocarbyl saccharide compound; optionally
   (c) a taurate surfactant; and optionally
   (d) one or more amphoteric or zwitterionic surfactants.

In some preferred embodiments the present invention provides a liquid laundry detergent composition comprising:
(a) at least one acyl alkyl isethionate surfactant of formula (I):

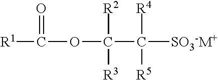

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation;
   (b) at least one hydrocarbyl saccharide compound; optionally
   (c) a taurate surfactant; and optionally
   (d) one or more amphoteric or zwitterionic surfactants.

In some preferred embodiments the present invention provides a laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; optionally
(c) an alkyl acyl taurate; and optionally
(d) an alkyl amido betaine surfactant.

In some preferred embodiments the present invention provides a solid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; optionally
(c) an alkyl acyl taurate; and optionally
(d) an alkyl amido betaine surfactant.

In some preferred embodiments the present invention provides a liquid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; optionally
(c) an alkyl acyl taurate; and optionally
(d) an alkyl amido betaine surfactant.

In some preferred embodiments the present invention provides a laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(c) an alkyl acyl taurate.

In some preferred embodiments the present invention provides a solid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(c) an alkyl acyl taurate.

In some preferred embodiments the present invention provides a liquid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(c) an alkyl acyl taurate.

In some preferred embodiments the present invention provides a laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(d) an alkyl amido betaine surfactant.

In some preferred embodiments the present invention provides a solid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(d) an alkyl amido betaine surfactant.

In some preferred embodiments the present invention provides a liquid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside; and
(d) an alkyl amido betaine surfactant.

In some preferred embodiments the present invention provides a laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside;
(c) an alkyl acyl taurate; and
(d) an alkyl amido betaine surfactant.

In some preferred embodiments the present invention provides a solid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside;
(c) an alkyl acyl taurate; and
(d) an alkyl amido betaine surfactant.

In some preferred embodiments the present invention provides a liquid laundry detergent composition comprising:
(a) an acyl alkyl isethionate surfactant selected from one or more of sodium lauroyl methyl isethionate, sodium cocoyl methyl isethionate and sodium oleoyl methyl isethionate.;
(b) an alkyl glucoside, preferably lauryl glucoside and/or decyl glucoside;
(c) an alkyl acyl taurate; and
(d) an alkyl amido betaine surfactant.

According to a second aspect of the present invention, there is provided a packaged detergent product comprising a container and a detergent composition according to the first aspect.

Preferred features of the second aspect are as defined in relation to the first aspect.

In the second aspect the detergent composition may be provided in loose powder or liquid form where a user measures out a dose each time before use.

In some preferred embodiments the packaged detergent composition comprises a plurality of unit doses of the detergent composition of the first aspect. In some embodiments the unit doses are provided in water-soluble film sachets, as discussed above. For example, suitable water-soluble film sachets may be formed from polyvinyl alcohol.

In preferred embodiments the detergent composition of the first aspect is provided in unit dose form.

The detergent compositions of the present invention have been found to provide excellent cleaning performance in the absence of sulfates, ethoxylated surfactants and linear and/or branched alkylbenzene sulfonate surfactants.

This offers environmental and health benefits.

The compositions of the present invention have been found to especially effective as laundry detergents.

According to a third aspect of the present invention, there is provided a method of laundering one or more items, the method comprising contacting the one or more items with a detergent composition comprising:
(a) at least one acyl alkyl isethionate surfactant of formula (I):

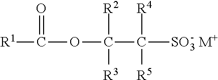

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation; and
(b) at least one hydrocarbyl saccharide compound.

Preferably the method of the third aspect involves the steps of loading the one or more items into a washing machine, adding the detergent composition into the washing machine, and running a wash cycle of the washing machine.

Preferred features of the third aspect are as defined in relation to the first and second aspects.

The detergent composition used in the third aspect is suitably as defined in the first aspect.

According to a fourth aspect of the present invention, there is provided the use of a detergent composition in toilet care, manual dishwashing, laundry, fabric care, kitchen care, carpet cleaning, vehicle care, polishing products, machine cleaning and maintenance, pesticides, insecticides, fungicides, herbicides, oilfield chemical applications, marine applications, personal care or institutional/industrial cleaning; wherein the detergent composition comprises:
(a) at least one acyl alkyl isethionate surfactant of formula (I):

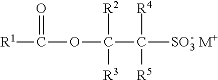

wherein R¹ represents an optionally substituted C₄-C₃₆ hydrocarbyl group;
each of R², R³, R⁴ and R⁵ independently represents hydrogen or a C₁-C₄ alkyl group and wherein at least one of R², R³, R⁴ and R⁵ is not hydrogen; and M⁺ represents a cation; and
(b) at least one hydrocarbyl saccharide compound.

Suitably the use of the fourth aspect provides improved cleaning performance, suitably in laundry applications.

Preferred features of the fourth aspect are as defined in relation to the first, second and third aspects.

The invention will now be further described with reference to the following non-limiting examples.

### Example 1

Compositions of example 1 were prepared comprising the following components:

TABLE 1

| | | | | 4 | | | |
|---|---|---|---|---|---|---|---|
| Composition | 1 | 2 | 3 | (comp) | 5 | 6 | 7 |
| SLMI (wt % active) | 14.50 | 7.25 | 4.83 | | | | |
| SCMI (wt % active) | | | | | 14.50 | 7.25 | 4.83 |
| APG (wt % active) | 3.50 | 3.50 | 3.50 | | 3.50 | 3.50 | 3.50 |
| MOT (wt % active) | | 7.25 | 4.83 | | | 7.25 | 4.83 |
| COT (wt % active) | | | 4.83 | | | | 4.83 |
| SLS (wt % active) | | | | 11.0 | | | |
| AE (wt % active) | | | | 7.00 | | | |
| Water | To 100% | To 100% | To 100% | To 100% | To 100% | To 100% | To 100% |
| Total surfactant | 18% | 18% | 18% | 18% | 18% | 18% | 18% |

SLMI: sodium lauroyl methyl isethionate - commercially available as a solid comprising 82 wt % active material
SCMI: sodium cocoyl methyl isethionate - commercially available as a solid comprising 82 wt % active material
APG: alkyl polyglucosides (decyl glucoside) - commercially available as a 50 wt % solution in water
MOT: sodium methyl oleoyl taurate - commercially available as a solid comprising 70 wt % active material
COT: sodium methyl cocoyl taurate - commercially available as a solid comprising 75 wt % active material
SLS: sodium lauryl sulfate - commercially available as a powder as a solid comprising 92 wt % active material
AE: alcohol ethoxylate (C12-15 Pareth-7) - commercially available as 100 wt % active material
Compositions 1 to 3 and 5 to 7 are of the invention. Composition 4 is comparative.

### Example 2

The compositions of example 1 were tested according to the following method which corresponds to a standard test ASTM D4265-21 modified for use in a tergotometer:

Soiled and clean cotton and cotton/polyester swatches were cut into 5 cm×5 cm squares. Compositions 1 to 7 from Example 1 were placed into separate tergotometer pots and the temperature of each pot was adjusted to approximately 100° F. (37.8° C.), before lowering the tergotometer paddles into each pot, and covering to retain the temperature. The tergotometer was set to 100 RPM, with reversal after every 50 revolutions. 8 soiled swatches and 4 clean swatches were added to each pot and washed for 30 minutes. After the 30-minute wash was complete, the water was drained and the cloths squeezed to remove excess water and set aside. The swatches were rinsed by refilling the tergotometer's pots with 1 Liter of water of the same hardness used during washing, and running at 100 RPM for 5 minutes. After rinsing, the water was drained and the cloths hand squeezed before laying flat to air dry, out of direct sunlight.

### Stain Removal Index (SRI):

A colorimeter was used to measure the reflectance of an unwashed, clean swatch (UF) (unwashed fabric samples can be used as references for all products tested).

Prior to washing, the reflectance of the unwashed soiled swatch (US) was measured.

After washing, rinsing and drying, the reflectance of the washed soiled swatch (WS) was measured The CIE L*, a*, b* color values from the reflectance measurements were determined.

The SRI may be calculated using the following equation:

$$SRI = \frac{100 \times {{Δ E}^{*}}_{( US - UF )} - {{Δ E}^{*}}_{( WS - UF )}}{{{Δ E}^{*}}_{( US - UF )}}$$

where:
US=Unwashed soiled swatch,
UF=Unwashed (clean) swatch,
WS=Washed soiled swatch.
ΔE*(US-UF)=Delta-E color difference between the unwashed soiled swatch and the unwashed clean swatch, and
ΔE*(WS-UF)=Delta-E color difference between the washed soiled swatch and the unwashed (clean) swatch.

The value delta E* is calculated as:

$${Δ E}^{*} = \sqrt{{Δ L}^{* 2} + {Δ α}^{* 2} + {Δ b}^{* 2}}$$

### Residual Stain Index (RSI):

The reflectance of the unwashed, clean swatch (UF) was measured (unwashed fabric samples can be used as references for all products tested).

After washing, rinsing and drying, the reflectance of the washed soiled swatches (WS) was measured.

The CIE L*, a*, b* color values from the reflectance measurements were determined.

RSI can be calculated using the equation:

$$RSI = 100 - {Δ E}_{( UF - WS )}^{*}$$

where:
UF=Unwashed (clean) swatch, and
WS=Washed soiled swatch.

The value Delta-E* is calculated as:

$${Δ E}^{*} = \sqrt{{Δ L}^{* 2} + {Δ α}^{* 2} + {Δ b}^{* 2}}$$

Test results for Compositions 1 to 4 for olive oil stains on cotton or blends of cotton and polyester are shown in table 2.

TABLE 2

| | | | | Av. Stain | Av. Residual |
|---|---|---|---|---|---|
| | | | | Removal | Stain Index |
| Composition | surfactants | Soil | Substrate | Index (SRI)* | (RSI)* |
| 1 | SLMI + APG | Olive Oil | Cotton | 11 | 69 |
| 1 | SLMI + APG | Olive Oil | Polyester/ | 19 | 71 |
| | | | Cotton blend | | |
| 2 | SLMI + APG + | Olive Oil | Cotton | 8.5 | 68 |
| | MOT | | | | |
| 2 | SLMI + APG + | Olive Oil | Polyester/ | 20.8 | 71.5 |
| | MOT | | Cotton blend | | |
| 3 | SLMI + APG + | Olive Oil | Cotton | 9 | 68.5 |
| | MOT + COT | | | | |
| 3 | SLMI + APG + | Olive Oil | Polyester/ | 21.6 | 71.8 |
| | MOT + COT | | Cotton blend | | |
| 4 | SLS + AE | Olive Oil | Cotton | 11 | 69.5 |
| (comparative) | | | | | |
| 4 | SLS + AE | Olive Oil | Polyester/ | 21.5 | 71.5 |
| (comparative) | | | Cotton blend | | |
| 5 | SLMI + APG | Olive Oil | Cotton | 8.49 | 61.67 |
| 6 | SLMI + APG + | Olive Oil | Cotton | 8.41 | 61.75 |
| | MOT | | | | |
| 7 | SLMI + APG + | Olive Oil | Cotton | 7.74 | 61.57 |
| | MOT + COT | | | | |

*Each test was performed in duplicate, and the results were averaged.

The test results show that the detergent compositions of the invention (compositions 1 to 3 and 5 to 7) provide comparative performance to compositions of the prior art comprising sulfates and ethoxylated compounds (composition 4).

### Example 3

Formulated solid laundry compositions were prepared comprising the ingredients listed in table 3:

TABLE 3

| Component | Composition | | | | |
|---|---|---|---|---|---|
| (wt % active) | 8 | 9 | 10 | 11 | 12 (comp) |
| Sodium sulfate | 35.3 | 33.8 | 33.8 | 35.3 | 41 |
| Sodium bicarbonate | 15 | 15 | 15 | 15 | 15 |
| Sodium carbonate | 10 | 10 | 10 | 10 | 10 |
| Sodium citrate | 10 | 10 | 10 | 10 | 10 |
| Sodium | 5 | 5 | 5 | 5 | 5 |
| percarbonate | | | | | |
| SLS | | | | | 11 |
| SLMI | | | | 14.5 | |
| SCMI | 14.5 | 7.2 | 4.8 | | |
| MOT | | 7.3 | 4.8 | | |
| COT | | | 4.8 | | |
| AE | | | | | 7 |
| Lauryl glucoside | | | | 3.5 | |
| Decyl glucoside | 3.5 | 3.5 | 3.5 | | |
| Total surfactant | 18 | 18 | 18 | 18 | 18 |
| (wt %) | | | | | |

The compositions were prepared by blending the dry components until homogenous, then slowly adding the liquid components while mixing. Samples were blended until homogenous.

Sodium sulfate, sodium bicarbonate, sodium carbonate, sodium citrate and sodium percarbonate were provided as powdered 100% active components.

SLS, SLMI, SCMI, MOT, COT and AE are as defined in example 1.

Lauryl glucoside and decyl glucoside are commercially available as a 50 wt % solution in water.

### Example 4

The compositions prepared in example 3 were dissolved in hard water (150 ppm calcium carbonate) at a dosage of 3.6 g/L and then tested according to the procedure set out in example 2. The results are in table 4:

TABLE 4

| Composition | Soil | Substrate | SRI | RSI |
|---|---|---|---|---|
| 8 | Olive oil | Cotton | 6.8 | 63.4 |
| 9 | Olive oil | Cotton | 11.1 | 64.8 |
| 10 | Olive oil | Cotton | 6.6 | 63.4 |
| 11 | Olive oil | Cotton | 6.1 | 63.1 |
| 12 | Olive oil | Cotton | 6.9 | 63.2 |
| 8 | Olive oil | Polyester/cotton | 15.9 | 70.1 |
| | | blend | | |
| 9 | Olive oil | Polyester/cotton | 18.5 | 71.4 |
| | | blend | | |
| 10 | Olive oil | Polyester/cotton | 22.6 | 71.8 |
| | | blend | | |
| 11 | Olive oil | Polyester/cotton | 19.4 | 70.7 |
| | | blend | | |
| 12 | Olive oil | Polyester/cotton | 29.2 | 74.4 |
| | | blend | | |

### Example 5

Formulated liquid laundry compositions were prepared comprising the ingredients listed in table 5:

TABLE 5

| Component | Composition | | | | |
|---|---|---|---|---|---|
| (wt % active) | 13 | 14 | 15 | 16 | 17 (comp) |
| Water | To 100% | To 100% | To 100% | To 100% | To 100% |
| MGDA | 0.78 | 0.78 | 0.78 | 0.78 | 0.78 |
| SLMI | 8 | 12 | 4 | 3 | |
| CAPB | 8 | 4 | 12 | 13 | |
| SLS | | | | | 11.5 |
| AE | | | | | 7 |
| Coco glucoside | 1 | 1 | 1 | 1 | |
| Total surfactant | 17% | 17% | 17% | 17% | 18.5% |
| Viscosity (cP) | ~65,000 | ~3500 | ~40,000 | ~17,000 | <100 |

The compositions were prepared by admixing the ingredients sequentially in the order listed in the table at room temperature with thorough mixing to ensure homogenisation before each addition.

SLMI, SLS and AE are as defined in example 1.

MGDA: methyl glycine diacetic acid-commercially available as a solid comprising 78 wt % active.

CAPB: cocoamido propyl betaine-commercially available as powder comprising 78 wt % active (compositions 13-15) or a solution comprising 34 wt % active (composition 16).

Coco glucoside: commercially available as an aqueous solution comprising 50 wt % active.

The viscosities of compositions 13 to 17 were measured with a Brookfield Viscosimeter at 25° C., Spindle 21-29, 10-50 RPM with a small sample adapter tool.

Each of compositions 13 to 16 is a clear isotropic viscous liquid.

### Example 6

The laundry washing performance of the compositions of table 5 was assessed using a Linitest procedure as described below on a panel of different stain types.

This Linitest procedure evaluates the ability of detergent formulations to remove applied soils from fabrics. Soiled fabric swatches are washed in a laboratory washer (Linitest, SDL Atlas) under specific conditions of temperature, detergent solution, washing time and water hardness. The Linitest machine rotates closed cannisters (8×500 mL) in a thermostatically controlled water bath at 40±2 RPM.

The washing conditions for the washing tests were as shown in Table 6:

TABLE 6

| Conditions | |
|---|---|
| Liquid Laundry Detergent Dosage (g/L) | 1 |
| Wash Time (min) | 30 |
| Temperature (° C.) | 30 |
| Water Hardness (ppm CaCO₃) | 300 |

For each run, seven of the same type of swatches were placed in each closed cannister within the Linitest washer. The runs were repeated four times. The swatches were each 6 cm×6 cm in size and simulate different types of fabrics and soil stains. Further details on the stain types of the different swatches and their product codes are given in Table 7. The swatches are commercially available from CFT Center for Test Materials (Vlaardingen, Netherlands).

TABLE 7

| Swatch | Fabric | Soil |
|---|---|---|
| A | Cotton | Mineral oil |
| B | Cotton | Olive oil |
| C | Polyester/cotton blend | Olive oil |
| D | Cotton | Sebum type A |
| E | Polyester/cotton blend | Sebum type A |
| F | Cotton | Sebum type B |
| G | Polyester/cotton blend | Sebum type B |

After the washing cycle, the washes liquors were emptied and the swatches rinsed under running cold tap water, then dried on a clothes line within a fume hood (in the dark and with the fume hood air flow switched on).

The effectiveness of the test detergent against a given type of swatch (e.g. specific fabric and stain type) was assessed by reflectometry. A Datacolor 400 high performance, reflectance only bench top spectrophotometer was used. The spectrophotometer had a D65 light source and a UV cut-off filter (400 nm). Reflectance measurements were taken by the spectrophotometer using the Y-value of the Y, x, y colour coordinates measurement (according to AISE protocol standard for laundry washing tests).

Measurements were taken of the soiled swatch before the wash and of the cleaned swatch after the wash. The measurements were taken with each swatch in a defined set of different positions and an average value used to generate the results. The results discussed herein were generated by averaging the measurements (before and after the wash) for the seven swatches used in each test. The following formula was used to calculate detergency as the percentage change in reflectance, compared to the initial reflectance before the washing cycle.

$$Detergency = \frac{100 \times ( {R}_{2} - {R}_{1} )}{{R}_{1}}$$

Where:
R1=reflectance of soiled cloth before wash
R2=reflectance of soiled cloth after wash

A positive value for the above detergency calculation means that the swatch material has become more reflective after washing. This signifies that the stain has been removed or partially removed from the swatch by the detergent composition during the test.

The results of these tests are provided in table 8.

The values in the table are the detergency score for each swatch and stain type.

TABLE 8

Composition

| Swatch | 13 | 14 | 15 | 16 | 17 (comp) |
|---|---|---|---|---|---|
| A | 10.49 | 6.82 | 5.18 | 6.94 | 6.64 |
| B | 9.51 | 7.96 | 9.31 | 8.86 | 7.09 |
| C | 16.17 | 16.00 | 21.21 | 21.84 | 18.10 |
| D | 6.70 | 6.25 | 4.88 | 7.43 | 3.77 |
| E | 9.59 | 8.80 | 9.33 | 10.24 | 10.15 |
| F | 11.43 | 10.80 | 11.32 | 10.99 | 12.50 |
| G | 20.89 | 21.83 | 28.41 | 27.31 | 28.35 |

1. A liquid detergent composition comprising:
(a) at least one acyl alkyl isethionate surfactant of formula (I): 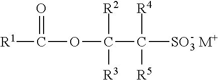
wherein R¹ represents an optionally substituted C₄-C₃₆ hydrocarbyl group;
each of R², R³, R⁴ and R⁵ independently represents hydrogen or a C₁-C₄ alkyl group and
wherein at least one of R², R³, R⁴ and R⁵ is not hydrogen; and M⁺ represents a cation; and
(b) at least one hydrocarbyl saccharide compound.

2. The liquid detergent composition according to claim 1, wherein component (a) comprises an acyl alkyl isethionate surfactant of formula (I) selected from one or more of sodium lauroyl methyl isethionate, sodium cocoyl methyl isethionate and sodium oleoyl methyl isethionate.

3. The liquid detergent composition according to claim 1, wherein component (b) comprises a compound of general formula (II): 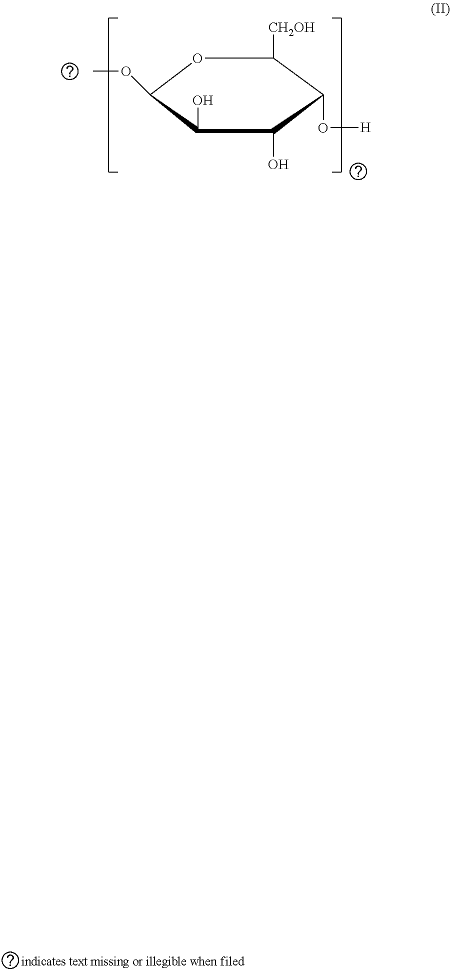
wherein n is from 2 to 24, preferably from 5 to 17, more preferably from 9 to 13 and m is from 1 to 10, preferably from 1 to 2.

4. The liquid detergent composition according to claim 1, further comprising (c) a taurate surfactant; preferably wherein component (c) comprises an alkyl acyl taurates selected from sodium lauroyl methyl taurate, sodium methyl myristoyl taurate, sodium methyl palmitoyl taurate, sodium methyl stearoyl taurate, sodium methyl cocoyl taurate, sodium methyl oleoyl taurate, and combinations thereof.

5. The liquid detergent composition according to claim 1, further comprising (d) one or more amphoteric or zwitterionic surfactants; preferably wherein component (d) comprises a betaine surfactant, preferably an alkyl amido betaine surfactant.

6. The liquid detergent composition according to claim 1 comprising less than 250 ppm 1,4-dioxane; and/or which comprises less than 1 wt % alkoxylated surfactants; and/or which comprises less than 1 wt % sulfate containing surfactants; and/or which comprises less than 1 wt % of linear and/or branched alkylbenzene sulfonates.

7. The liquid detergent composition according to claim 1, wherein component (a), component (b) and, when present, component (c) and/or component (d) together make up at least 80 wt % together make up at least 80 wt % of all surfactant compounds present in the composition.

8. (canceled)

9. The liquid detergent composition according to claim 1, which is a laundry composition.

10. The liquid detergent composition according to claim 1, comprising one or more components selected from include chelating agents, fillers, builders, bleaching agents, bleach activators, pigments, dispersants, polymeric dispersing agents, dyes, dye transfer inhibitors, fragrances, fragrance delivery systems, enzyme stabilizers, biocides, probiotics, preservatives, pH adjusting agents, phosphates, silicates, zeolites, peroxide based compounds (especially hydrogen peroxide, chlorine bleaches), perborate compounds, percarbonate compounds, bleach activators and catalysts, cationic surfactants, redeposition additives, brighteners, sud suppressors, fabric softeners and hydrotropes.

11. The liquid detergent composition according to claim 1, which is in unit dose form.

12. The liquid detergent composition according to claim 1, comprising less than 25 wt % surfactant compounds in total, based on the total weight of the composition, preferably less than 22 wt % surfactant compounds in total.

13. A packaged detergent product comprising a container and a liquid detergent composition according to claim 1.

14. A method of laundering one or more items, the method comprising contacting the one or more items with a liquid detergent composition comprising: (a) at least one acyl alkyl isethionate surfactant of formula (I): 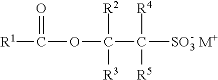
wherein R¹ represents an optionally substituted C₄-C₃₆ hydrocarbyl group;
each of R², R³, R⁴ and R⁵ independently represents hydrogen or a C₁-C₄ alkyl group and
wherein at least one of R², R³, R⁴ and R⁵ is not hydrogen; and M⁺ represents a cation; and
(b) at least one hydrocarbyl saccharide compound.

15. The method according to claim 14 which involves the steps of loading the one or more items into a washing machine, adding the liquid detergent composition into the washing machine, and running a wash cycle of the washing machine.

16. The use of a liquid detergent composition in toilet care, manual dishwashing, laundry, fabric care, kitchen care, carpet cleaning, vehicle care, polishing products, machine cleaning and maintenance, pesticides, insecticides, fungicides, herbicides, oilfield chemical applications, marine applications, personal care or institutional/industrial cleaning; wherein the detergent composition comprises:
(a) at least one acyl alkyl isethionate surfactant of formula (I): 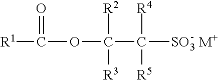
wherein R¹ represents an optionally substituted C₄-C₃₆ hydrocarbyl group;
each of R², R³, R⁴ and R⁵ independently represents hydrogen or a C₁-C₄ alkyl group and
wherein at least one of R², R³, R⁴ and R⁵ is not hydrogen; and M⁺ represents a cation; and
(b) at least one hydrocarbyl saccharide compound.

17. The use according to claim 16 to provide improved cleaning performance in laundry applications.
